(19) United States
(12) Patent Application Publication
(10) Pub. No.: US 2010/0125212 A1
(43) Pub. Date: May 20, 2010

(54) METHOD AND APPARATUS FOR TESTING ACCURACY OF BLOOD PRESSURE MONITORING APPARATUS

(75) Inventors: Seok Chan KIM, Seoul (KR); Jong Pal KIM, Seoul (KR); Kun-soo SHIN, Seongnam-si (KR); Sang-kon BAE, Seongnam-si (KR); Kyoung-ho KANG, Hwaseong-si (KR); Youn-ho KIM, Hwaseong-si (KR)

Correspondence Address: CANTOR COLBURN, LLP, 20 Church Street, 22nd Floor, Hartford, CT, 06103, US

(73) Assignee: SAMSUNG ELECTRONICS CO., LTD., Suwon-si (KR)

(21) Appl. No.: 12/581,283

(22) Filed: Oct. 19, 2009

(30) Foreign Application Priority Data
Nov. 17, 2008 (KR) 10-2008-0114065

(51) Int. Cl. A61B 5/021; G01L 27/00

(52) U.S. Cl. 600/485; 73/1.57

(57) ABSTRACT

A method for testing accuracy of blood pressure measurement in a blood pressure monitoring apparatus includes calculating a difference between measured blood pressures of a user measured at two or more measurement points, calculating a difference between hydrostatic pressures of blood estimated at the two or more measurement points, and calculating an error of the measured blood pressures.

### CROSS-REFERENCE TO RELATED APPLICATION

This application claims priority to Korean Patent Application No. 10-2008-0114065, filed on Nov. 17, 2008, and all the benefits accruing therefrom under 35 U.S.C. §119, the contents of which in its entirety are herein incorporated by reference.

### BACKGROUND

1) Field

The following description relates to a method and apparatus for testing accuracy of a blood pressure monitoring apparatus.

2) Description of the Related Art

Patients suffering from chronic diseases are increasing in number. For example, in 2008 78 million people were suffering from chronic diseases in the United States. Accordingly, there is increasing concern about chronic diseases. Typical chronic diseases include diabetes, hypertension, cardiovascular diseases and lung diseases, for example. Continuous monitoring of various vital signs is often required for patients with chronic diseases. Specifically, blood pressure is typically used as one indices of a patient's health condition. Thus, blood pressure measurement apparatuses are commonly used in medical institutions and homes. To ensure that these blood pressure measurement apparatuses meet safety and performance requirements, the United States Food and Drug Administration (“US FDA”) requires that standards for approval of blood pressure measurement apparatuses comply with requirements of the Association for the Advancement of Medical Instrumentation (“AAMI”). More specifically, for example, American National Standards Institute (“ANSI”)/AAMI SP10, issued by AAMI, provides specification details, as well as safety and performance requirements, for blood pressure measurement apparatuses.

To verify whether the abovementioned standards are met, there is a need for an apparatus and method of testing the accuracy of blood pressure monitoring apparatuses.

### SUMMARY

Provided are a method and apparatus for testing accuracy of a blood pressure monitoring apparatus to substantially raise a reliability of blood pressure measurement results obtained therewith. In addition, a method and apparatus for testing accuracy of a blood pressure monitoring apparatus further determine and report to a user whether the blood pressure monitoring apparatus should be adjusted, so that the blood pressure monitoring apparatus is corrected when required.

Provided are a method of testing accuracy of blood pressure measurement in a blood pressure monitoring apparatus includes calculating a difference between measured blood pressures of a user measured at two or more measurement points, calculating a difference between hydrostatic pressures of blood estimated at the two or more measurement points based on a height difference between the measurement points and blood density, and calculating an error in the measured blood pressures based on the difference between the measured blood pressures and the difference between the hydrostatic pressures of blood.

Provided is a computer program product including a computer readable computer program code for implementing a method of testing accuracy of blood pressure measurement in a blood pressure monitoring apparatus, and instructions for causing a computer to implement the method. The method includes calculating a difference between measured blood pressures of a user measured at two or more measurement points, calculating a difference between hydrostatic pressures of blood estimated at the two or more measurement points based on a height difference between the two or more measurement points and a blood density, and calculating an error of the measured blood pressures based on the difference between the measured blood pressures and the difference between the hydrostatic pressures of blood.

Provided is a blood pressure monitoring apparatus including: a blood pressure measurement unit which measures blood pressures of a user at two or more measurement points to generate measured blood pressure; a blood pressure difference calculation unit which calculates a difference between the measured blood pressures; a hydrostatic pressure difference calculation unit which calculates a difference between hydrostatic pressures of blood estimated at the two or more measurement points; an error calculation unit which calculates an error based on the difference between the measured blood pressures and the difference between the hydrostatic pressures of blood; and a user interface unit which reports the error to the user.

### BRIEF DESCRIPTION OF THE DRAWINGS

The above and/or other aspects, features and advantages of the present invention will become more readily apparent by describing in further detail exemplary embodiments thereof with reference to the accompanying drawings, in which:

FIG. 1 is a block diagram of an exemplary embodiment of a blood pressure monitoring apparatus according to the present invention;

FIGS. 2A and 2B illustrate an exemplary embodiment of a method of measuring blood pressure of a user using an exemplary embodiment of a blood pressure measurement unit in the blood pressure monitoring apparatus of FIG. 1;

FIGS. 3A and 3B illustrate an exemplary embodiment of a method of measuring blood pressure at two measurement points having a height difference equivalent to a distance between a wrist and elbow of a user according to the present invention;

FIG. 4 is an exemplary embodiment of a user interface unit in the blood pressure monitoring apparatus of FIG. 1;

FIG. 5 is a block diagram of an exemplary embodiment of a calculation unit of the blood pressure monitoring apparatus of FIG. 1;

FIG. 6 illustrates exemplary embodiments of methods of acquiring a height difference using physical sizes of a user according to the present invention;

FIG. 7 illustrates an exemplary embodiment of a method of calculating a difference between estimated hydrostatic pressures according to the present invention;

FIG. 8 is a flowchart illustrating an exemplary embodiment of a method of testing an accuracy of the blood pressure monitoring apparatus of FIG. 1; and

FIG. 9 is a flowchart illustrating an alternative exemplary embodiment of a method of testing an accuracy of the blood pressure monitoring apparatus of FIG. 1.

### DETAILED DESCRIPTION

The general inventive concept will now be described more fully hereinafter with reference to the accompanying drawings, in which exemplary embodiments are shown. The present invention may, however, be embodied in many different forms and should not be construed as limited to the embodiments set forth herein. Rather, these embodiments are provided so that this disclosure will be thorough and complete, and will fully convey the scope of the invention to those skilled in the art. Like reference numerals refer to like elements throughout.

It will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that although the terms “first,” “second,” “third” etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the present invention.

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising,” or “includes” and/or “including,” when used in this specification, specify the presence of stated features, regions, integers, steps, operations, elements and/or components, but do not preclude the presence or addition of one or more other features, regions, integers, steps, operations, elements, components and/or groups thereof.

Furthermore, relative terms, such as “lower” or “bottom” and “upper” or “top” may be used herein to describe one element's relationship to other elements as illustrated in the Figures. It will be understood that relative terms are intended to encompass different orientations of the device in addition to the orientation depicted in the Figures. For example, if the device in one of the figures is turned over, elements described as being on the “lower” side of other elements would then be oriented on the “upper” side of the other elements. The exemplary term “lower” can, therefore, encompass both an orientation of “lower” and “upper,” depending upon the particular orientation of the figure. Similarly, if the device in one of the figures were turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which the present invention belongs. It will be further understood that terms, such as those defined in commonly used dictionaries, should be interpreted as having a meaning which is consistent with their meaning in the context of the relevant art and the present disclosure, and will not be interpreted in an idealized or overly formal sense unless expressly so defined herein.

Exemplary embodiments of the present invention are described herein with reference to cross section illustrations which are schematic illustrations of idealized embodiments of the present invention. As such, variations from the shapes of the illustrations as a result, for example, of manufacturing techniques and/or tolerances, are to be expected. Thus, embodiments of the present invention should not be construed as limited to the particular shapes of regions illustrated herein but are to include deviations in shapes which result, for example, from manufacturing. For example, a region illustrated or described as flat may, typically, have rough and/or nonlinear features. Moreover, sharp angles which are illustrated may be rounded. Thus, the regions illustrated in the figures are schematic in nature and their shapes are not intended to illustrate the precise shape of a region and are not intended to limit the scope of the present invention.

Hereinafter, exemplary embodiments of the present invention will be described in further detail with reference to the accompanying drawings.

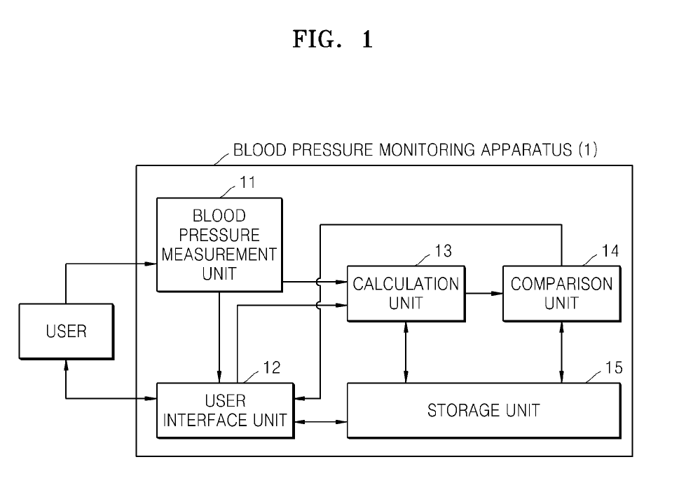

FIG. 1 is a block diagram of an exemplary embodiment of a blood pressure monitoring apparatus according to the present invention, and, more specifically, FIG. 1 is a block diagram of an exemplary embodiment of a blood pressure monitoring apparatus which provides, but is not limited to, a function of testing an accuracy of a blood pressure measurement.

An exemplary embodiment of a blood pressure monitoring apparatus 1 includes a blood pressure measurement unit 11, a user interface unit 12, a calculation unit 13, a comparison unit 14 and a storage unit 15.

The blood pressure monitoring apparatus 1 according to an exemplary embodiment may be, for example, any appliance, including, but not limited to, blood pressure meters, blood pressure measurement devices and hemadynamometers. In addition, specific examples of different types of hemadynamometers include sphygmomanometers and automatic blood pressure monitors, for example. Furthermore, sphygmomanometers include, for example, a mercurial type, an aneroid type and a stand type, but alternative exemplary embodiments are not limited thereto. Automatic blood pressure monitors include an upper arm type, a wrist type and a finger type, for example, based on a target measurement point, e.g., a point where blood pressure is measured. Thus, it will be apparent to those of ordinary skill in the art that the blood pressure monitoring apparatus 1 according to an exemplary embodiment conceptually applies to, but is not limited by, the aforementioned blood pressure measurement devices.

The blood pressure measurement unit 11 measures blood pressure of a user using a direct and/or indirect method, an invasive and/or noninvasive method, and/or an intrusive and/or nonintrusive method, for example. As used herein, “blood pressure” indicates a pressure of blood acting on a wall of blood vessels as blood pumped out of a heart flows along the blood vessels. In addition, blood pressure includes, but is not limited to, arterial blood pressure, capillary blood pressure and vein blood pressure, based on a type of blood vessel where blood pressure is measured. For example, arterial blood pressure varies according to heartbeats. In addition, blood pressure includes a systolic pressure, e.g., when blood flows into an artery as ventricles of the heart contract, and a diastolic pressure, e.g., a pressure acting against the arterial wall, due to elasticity of the arterial wall, when the ventricles expand and the blood stays in the ventricles. Accordingly, the blood pressure measurement unit 11 according to an exemplary embodiment measures at least one of the systolic pressure, the diastolic pressure and an average blood pressure of a user, but alternative exemplary embodiments are not limited thereto.

More specifically with respect to the abovementioned blood pressure measurement methods, the direct method includes directly inserting a catheter into the carotid arteries, for example, and connecting the catheter to a manometer to measure blood pressure. The indirect method includes winding a cuff around an upper portion of a patient, e.g., a user's, arm, pumping air into the cuff to compress the upper arm and measuring blood pressure when flow of blood in a brachial artery changes, e.g., when blood stops and/or starts flowing. The invasive method measures blood pressure when a catheter is directly inserted into a blood vessel. In contrast, the noninvasive method measures blood pressure without inserting anything into the blood vessel. Similarly, the intrusive method uses a cuff, while the nonintrusive method is a cuffless blood pressure measurement method.

Although the catheter to be directly inserted into the blood vessel in the invasive method, the invasive method allows continuous and accurate measurement of blood pressure.

Noninvasive methods include an auscultatory method of measuring blood pressure using Korotkoff sounds, an oscillometry method using vibration generated due to flow of blood, a method using a tonometer, and a method using pulse transit time (“PTT”), for example. More specifically, in the auscultatory method and the oscillometry method, the cuff expands and contract, and these methods are thereby intrusive and cannot continuously measure blood pressure. Although the method using a tonometer continuously measures blood pressure, the tonometer is a sensitive instrument. The method using PTT uses a time interval between an R-wave from electrocardiography (“ECG”) and a peak from photoplethysmography (“PPG”), and has invasive and nonintrusive characteristics, and thereby continuously measures blood pressure. It will be noted by those of ordinary skill in the art that the abovementioned methods of measuring blood pressure are applicable to the blood pressure measurement unit 11 according to an exemplary embodiment, and that alternative exemplary embodiments are not limited thereto. Therefore, exemplary embodiments are applicable to all blood pressure monitoring apparatuses and accurately test an accuracy of any blood pressure monitoring apparatus without requiring any additional equipment.

The blood pressure measurement unit 11 according to an exemplary embodiment measures blood pressures of the user at different measurement points, e.g., multiple measurement points and, specifically, more than one measurement point. More specifically, multiple measurement points includes at least two measurement points, e.g., two or more measurement points, which are determined according to the user's selection or, alternatively, are based on characteristics of the blood pressure monitoring apparatus 1 according to the particular exemplary embodiment associated therewith.

In general as a number of measurement points where blood pressure is measured increases, a reliability of the accuracy test of the blood pressure monitoring apparatus 1 increases, e.g., improves. However, for purposes of description herein, the number of measurement points is two, but this is for purposes of convenience of explanation only. It will be noted by those of ordinary skill in the art that an accuracy of the blood pressure monitoring apparatus 1 according to an exemplary embodiment can be tested using results measured at more than two measurement points. In addition, the number of measurement points where the blood pressure is measured may be determined according to the user's selection. Regardless, when the blood pressure measurement unit 11 is located at a height greater than a height of the user's heart, reliability of the results of the accuracy test in the blood pressure monitoring apparatus 1 is improved.

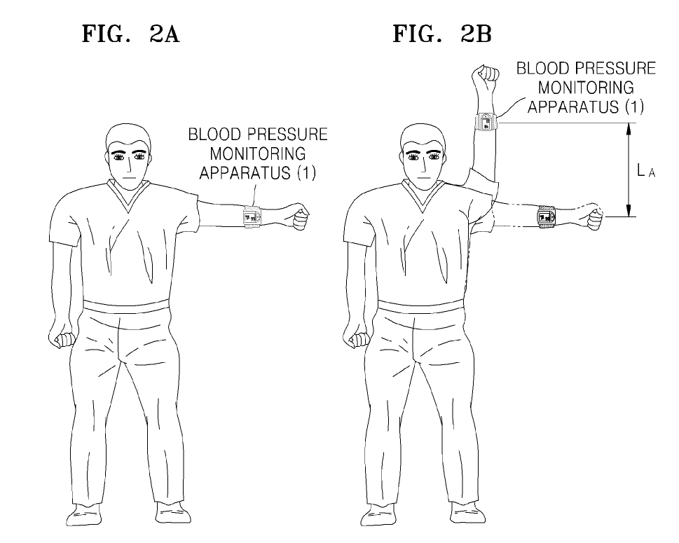

FIGS. 2A and 2B illustrate an exemplary embodiment of a method of measuring blood pressure of a user using an exemplary embodiment of a blood pressure measurement unit of the blood pressure monitoring apparatus 1 of FIG. 1 and, more particularly, FIGS. 2A and 2B illustrate an exemplary embodiment of a blood pressure measurement unit 11 that uses a wrist-type automatic blood pressure measurement method. More specifically, FIG. 2A illustrates an exemplary embodiment of a method of measuring blood pressure wherein the user horizontally stretches their arm straight out to be substantially parallel with their shoulder, while FIG. 2B illustrates an exemplary embodiment of a method of measuring blood pressure wherein the user raises their arm straight up. Thus according to the exemplary embodiments of the methods mentioned above and described in further detail below, the blood pressure of the user can be measured at two measurement points having a height difference therebetween. In an exemplary embodiment, the height difference is measured substantially vertically, e.g., in a direction of a gravitational force on the user. In an exemplary embodiment, the height difference is equivalent to a distance L_{A}, measured from a wrist to a shoulder of the user, as shown in FIG. 2B.

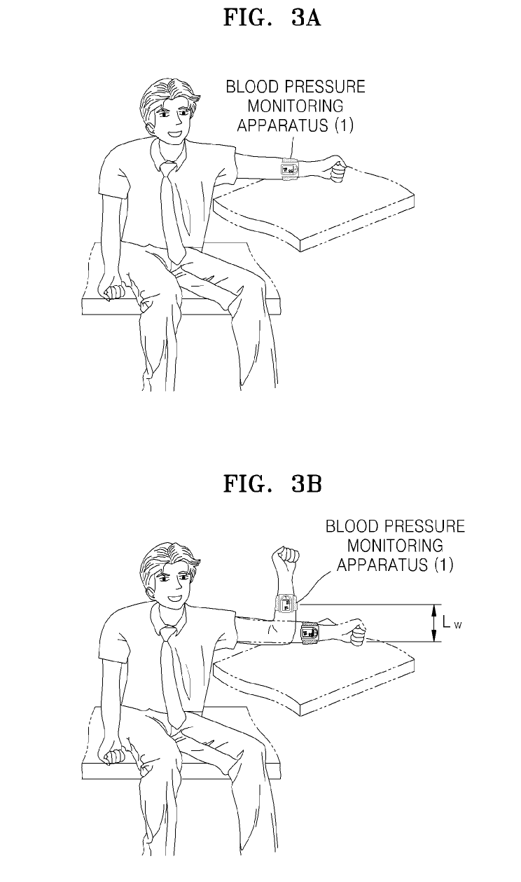

FIGS. 3A and 3B illustrate an exemplary embodiment of a method of measuring blood pressure at two measurement points having a height difference equivalent to a distance between a wrist and elbow of the user. More specifically, FIG. 3A illustrates an exemplary embodiment of a method of measuring blood pressure wherein the user is seated and stretches out their arm to a height of their shoulder, while FIG. 3B illustrates an exemplary embodiment of a method of measuring blood pressure wherein the user is seated and bends their elbow to raise their wrist up in a direction substantially parallel to the a direction of gravity. According to the methods mentioned above and described in further detail below, the blood pressure of the user can be measured at two measurement points, e.g., a first measurement point and a second measurement point, having a height difference in the direction of gravity therebetween. In an exemplary embodiment, for example, the height difference is a distance L_{w}, from a wrist to an elbow of the user, as shown in FIG. 3B. The exemplary embodiments of the blood pressure measurement methods illustrated in FIGS. 2A and 2B, as well as in FIGS. 3A and 3B, are not limited to the foregoing description, and alternative exemplary embodiments may include variations thereof. In addition, as described above, alternative exemplary embodiments include measuring blood pressures at more than two measurement points.

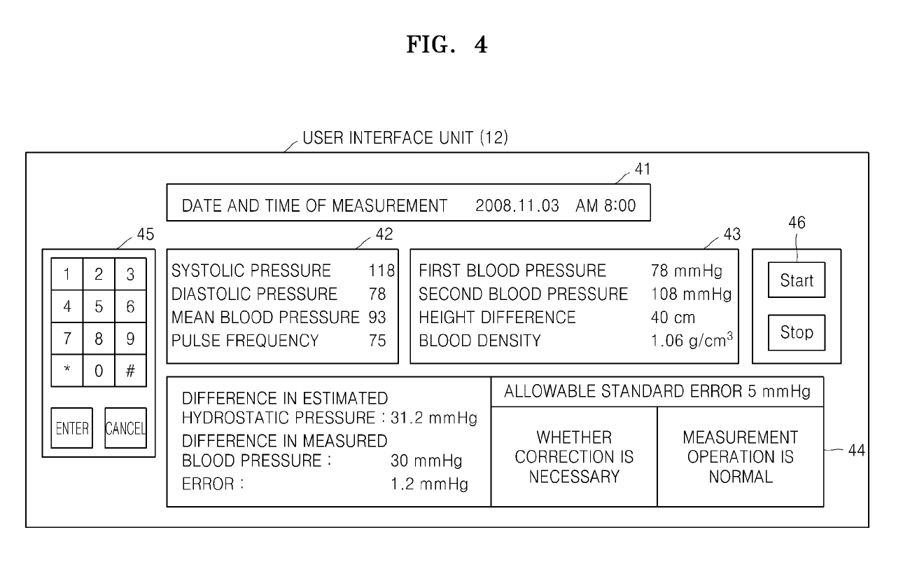

Referring to FIG. 4, which is an exemplary embodiment of the user interface unit 12 of the blood pressure monitoring apparatus 1 shown in FIG. 1, the user interface unit 12 receives information such as a blood density, a height difference, an allowable standard error and a physical size of the user, for example, from the user, and displays information about measured blood pressure results, hydrostatic pressure difference calculation results, blood pressure difference calculation results, error calculation results and whether correction is required, for example. The user interface unit 12 acquires information from the user, for example, using any type of suitable information input device or method, such as a keyboard, a mouse, a touch screen and speech recognition, for example. The blood pressure monitoring apparatus 1 according to an exemplary embodiment acquires, through the user interface unit 12, information such as a height difference between measurement points where blood pressures have been measured, blood density, and an information indication method, for example, which depend on the user's selection and/or a setting of the blood pressure monitoring apparatus 1. In addition, the user interface unit 12 includes devices which display visual information, such as a display, a liquid crystal display (“LCD”) screen, a light-emitting-diode (“LED”), and a division display device, for example, and devices providing auditory information, e.g., sound, such as speakers, for example, to the user.

Referring now to FIGS. 1 and 4, the user wears the blood pressure monitoring apparatus 1 and presses a start button 46 to measure blood pressure. The user interface unit 12 displays, for example, a date and time of a blood pressure measurement 41, measured blood pressure results 42 and blood pressures measured at multiple measurement points 43. Although the exemplary embodiment shown in FIGS. 1 and 4 and described herein with reference to blood pressures measured at two measurement points for convenience of explanation, it will be noted that alternative exemplary embodiments are not limited thereto. Rather, blood pressures measured at multiple measurement points, e.g., more than two measurement points, may be displayed along with a height difference between the multiple measurement points in a blood pressure monitoring apparatus 1 according to an alternative exemplary embodiment.

In an exemplary embodiment, a height difference, a blood density and an allowable standard error, for example, may be acquired from the user through an input device 45. The user interface unit 12 displays the measured blood pressure results 42 and/or whether the blood pressure monitoring apparatus 1 operates normally in user interface unit part 44. In an exemplary embodiment, a method of displaying information may be selected by the user. It will be apparent to those of ordinary skill in the art that the user interface unit 12 according to alternative exemplary embodiments may use various methods to display information, such as using a touch screen, voice recognition and/or voice information, but alternative exemplary embodiments are not limited thereto.

Referring again to FIG. 1, the calculation unit 13 acquires the blood pressures measured in the blood pressure measurement unit 11, information input through the user interface unit 12, information stored in the storage unit 15, and calculates a difference between hydrostatic pressures, a difference between measured blood pressures and an error.

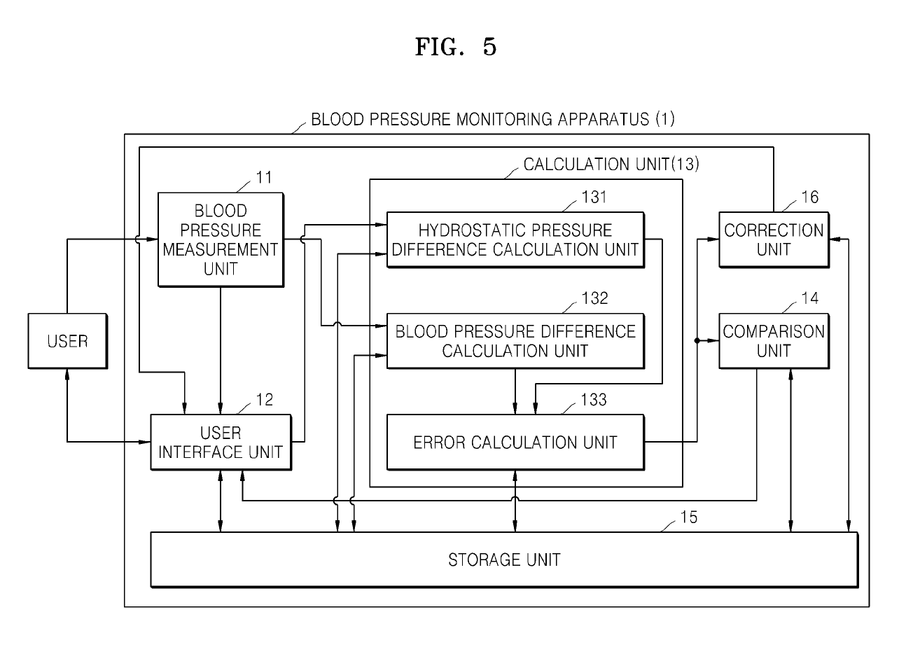

FIG. 5 is a block diagram of an exemplary embodiment of the calculation unit 13 of the blood pressure monitoring apparatus 1 shown in FIG. 1 and, moreover, FIG. 5 is an exemplary embodiment of the calculation unit 13 that tests an accuracy in blood pressure measurement according to the present invention. Referring to FIG. 5, the calculation unit 13 calculates a difference between hydrostatic pressures, a difference between measured blood pressures and an error based on the blood pressures measured in the blood pressure measurement unit 11, information input through the user interface unit 12, a height difference between the measurement points acquired by a height difference recognition sensor (not shown) and information stored in the storage unit 15, for example. The calculation unit 13 according to an exemplary embodiment includes a hydrostatic pressure difference calculation unit 131, a blood pressure difference calculation unit 132 and an error calculation unit 133, each of which will be described in further detail below.

In an exemplary embodiment, the hydrostatic pressure difference calculation unit 131 calculates a difference between the hydrostatic pressures of blood, measured at the multiple measurement points where the blood pressures are measured by the blood pressure measurement unit 11, based on information inputted via the user interface unit 12, information stored in the storage unit 15, and/or information acquired from the height difference recognition sensor. As used herein, “hydrostatic pressure” indicates a pressure acting on a static fluid. More particularly, the hydrostatic pressure of blood indicates a pressure of blood pushing against a blood vessel wall in response to a user's heartbeat. Although a waveform representing blood pressure varies, since blood in the human body is not a static fluid, when calculating the difference between the hydrostatic pressures of blood in an exemplary embodiment, the systolic and/or the diastolic pressure may be regarded as a static pressure at a point of time of measuring the blood pressure. In addition, a mean arterial pressure (“MAP”) of a measurement interval is substantially constant, and thus is regarded as a substantially static pressure. As used herein, the difference between the hydrostatic pressures of blood means a difference in pressure according to a height difference between multiple blood pressure measurement points having different heights. The difference between the hydrostatic pressures occurs due to a weight of blood and the height difference between the measurement points. In an exemplary embodiment, the difference between the hydrostatic pressures indicates a difference between hydrostatic pressures of blood at two measurement points where the blood pressures are measured. Therefore, in an exemplary embodiment, the difference between the hydrostatic pressures is a theoretical value acquired by calculation (described in greater detail below), and will therefore be referred to as a difference between estimated hydrostatic pressures.

In an exemplary embodiment, the hydrostatic pressure difference calculation unit 131 calculates the difference between the estimated hydrostatic pressures at the multiple measurement points where the blood pressures are measured by the user. The difference between the estimated hydrostatic pressures is calculated by multiplying a height difference, a blood density and an acceleration due to gravity. The hydrostatic pressure difference calculation unit 131 acquires the height difference between the multiple measurement points at which the blood pressures of the user are measured, from the user interface unit 12, the storage unit 15 and/or the height difference recognition sensor (not shown). A particular method of acquiring the height difference is determined according to the user's selection or a setting of the blood pressure monitoring apparatus 1, for example. The difference between the hydrostatic pressures is calculated as the difference between the hydrostatic pressures at the multiple measurement points where the blood pressures are measured by the blood pressure measurement unit 11. In an exemplary embodiment wherein the number of measurement points is more than two, two suitable arbitrary measurement points among the multiple measurement points are selected, and the difference between the hydrostatic pressures at the two measurement points is calculated and compared with the difference between the blood pressures measured at those measurement points to calculate an error. Alternatively, differences between the hydrostatic pressures at two of all the measurement points where the blood pressures are measured are calculated and stored in the storage unit 15, and then a difference between the hydrostatic pressures and a difference between the blood pressures measured at two different measurement points are compared to calculate errors. Thus, an operation of calculating errors is repeated for all of the multiple measurement points. As a result, a reliability of a method of testing the accuracy of the blood pressure monitoring apparatus 1 according to an exemplary embodiment is substantially improved. However, for purposes of convenience of explanation only, the description hereinafter will be described with reference to measuring blood pressure at only two measurement points having a height difference therebetween.

In an exemplary embodiment, a height difference between multiple measurement points where blood pressures are measured may be acquired by using various methods, such as from user input or a height difference recognition sensor, for example, but alternative exemplary embodiments are not limited thereto. In addition, the height difference may be estimated based on a physical size of the user. When a height difference input by the user is used, the hydrostatic pressure difference calculation unit 131 acquires the height difference input through the user interface unit 12. Thus, after blood pressures are measured on the two measurement points having the height difference therebetween, the user inputs the height difference through the user interface unit 12, and the hydrostatic pressure difference calculation unit 131 thereby acquires the input height difference. In an exemplary embodiment wherein blood pressures are measured at two measurement points having a height difference of 15 cm, for example, information indicating the height difference of 15 cm is inputted by the user through the user interface unit 12. More specifically, methods of inputting the height difference information may include keyboard and a voice recognition method, for example, but alternative exemplary embodiments are not limited thereto.

The hydrostatic pressure difference calculation unit 131 may acquire the height difference from a height difference recognition sensor (not shown). In an exemplary embodiment, the height difference recognition sensor is disposed on, e.g., is attached to, the blood pressure monitoring apparatus 1 and senses the height difference between the measurement points where the blood pressures are measured, and the hydrostatic pressure difference calculation unit 131 thereafter acquires information about the sensed height difference. The user interface unit 12 provides the height difference information to the user when measuring the blood pressures. For example, in an exemplary embodiment wherein blood pressures at two measurement points having a height difference are measured, after blood pressure has been measured at a first measurement point, and then when measuring blood pressure at a second measurement point begins, the user interface unit 12 may provide the height difference information by using a visual method (such as by displaying a message “The current height difference is 20 cm,” for example) and/or by an acoustic method (such as by outputting through a speaker a voice message indicating “the current height difference is 20 cm,” for example), thereby conveniently providing the height difference to the user.

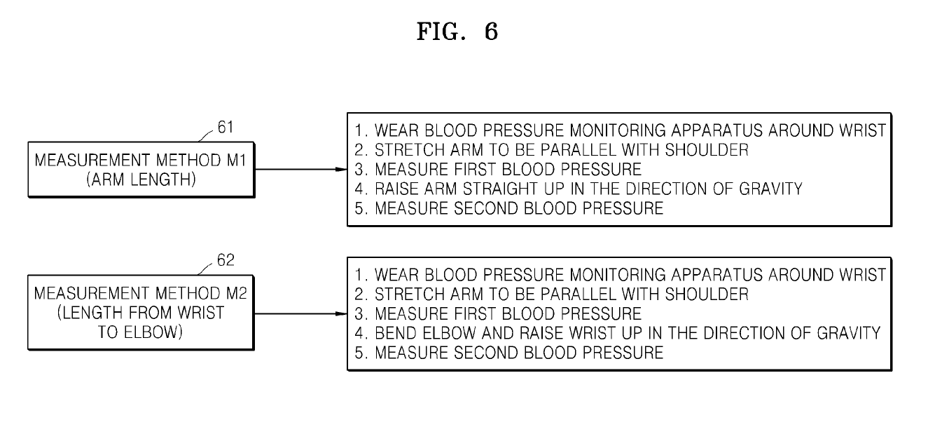

FIG. 6 illustrates exemplary embodiments of methods of acquiring a height difference using physical sizes of a user. Specifically, the user inputs their physical sizes through the user interface unit 12 of the blood pressure monitoring apparatus 1 (FIG. 5), and the blood pressure monitoring apparatus 1 estimates the height difference based on information corresponding to the input physical sizes of the user.

More specifically, information including a user's height, arm length and/or length from a wrist to an elbow, for example, is inputted by the user and stored in the storage unit 15 (FIG. 5) of the blood pressure monitoring apparatus 1. The user selects one of two measurement methods, e.g., either a first measurement method (“M1”) 61 or a second measurement method (“M2”) 62, as shown in FIG. 6, via the user interface unit 12 (FIG. 5). The blood pressure monitoring apparatus 1 thereby displays, through the user interface unit 12, guide information for measuring blood pressures at two measurement points having a height difference according to the measurement method selected by the user. As described in greater detail above, the user interface unit 12 may display the guide information by using a visual method, for example, or may reproduce the guide information by using an auditory method such as a voice signal, for example.

In an exemplary embodiment, for example, when the user selects the second measurement method 62, a length from the user's wrist to elbow is estimated, based on previously inputted physical sizes of the user, and the hydrostatic pressure difference calculation unit 131 (FIG. 5) uses the length from the user's wrist to elbow as the height difference. The user interface unit 12 may provide, in audio and/or visual form, information and/or instructions, such as “Please wear the blood pressure monitoring apparatus 1 on your wrist,” “Please stretch your arm to be parallel with your shoulder,” “Your blood pressure is being measured for the first time,” “Please bend your elbow for your wrist to be raised up in the direction of gravity,” and “Your blood pressure is being measured for the second time,” for example.

In addition, the user may input their physical size, such as their height, for example, via the user interface unit 12 of the blood pressure monitoring apparatus 1, and the blood pressure monitoring apparatus 1 thereby estimates the height difference based on the input physical size information, e.g., the user's height. In addition, when the user inputs their height and gender, for example, the blood pressure monitoring apparatus 1 calculates a difference between the hydrostatic pressures using stored information regarding average arm lengths or average lengths from the wrist to the elbow of people corresponding to the user height as the height difference.

Referring again to FIG. 5, the hydrostatic pressure difference calculation unit 131 acquires a blood density stored in the storage unit 15 or, alternatively, a blood density inputted through the user interface unit 12 by the user. A person's blood density is typically about 1.06 g/cm³, but this value may be corrected according to the user's instructions. Specifically, the hydrostatic pressure difference calculation unit 131 uses a blood density of 1.06 g/cm³ as a default setting. However, if the user selects a different blood density level, the different blood density level, inputted through the user interface unit 12 by the user, for example, may be used as described above.

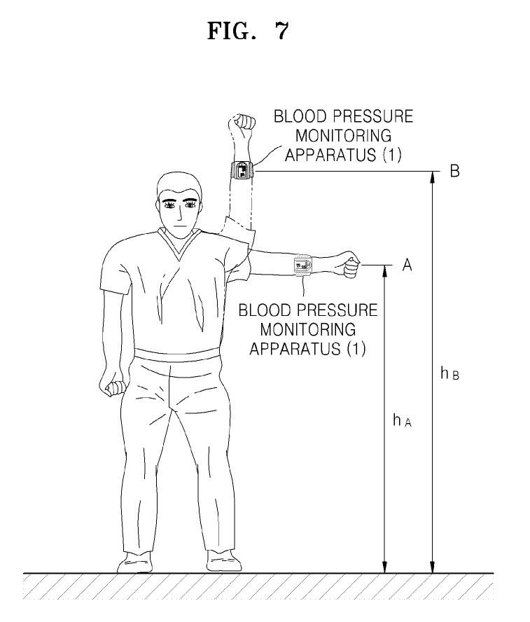

FIG. 7 illustrates an exemplary embodiment of a method of calculating a difference between estimated hydrostatic pressures according to the present invention. In general, a person's bloodstream includes potential energy, pressure energy and kinetic energy. In addition, for a fluid having a constant density, a sum of the potential energy, the pressure energy and the kinetic energy is constant. Bernoulli's theorem numerically defines a relationship between a flow rate and pressure of a fluid, e.g., blood, based on the principle of the conservation of energy, as expressed in Equation (1) below.

$$\frac{{υ}^{2}}{2 g} + \frac{{P}_{A}}{ρ g} + {h}_{A} = \frac{{υ}^{2}}{2 g} + \frac{{P}_{B}}{ρ g} + {h}_{B} = const . \qquad Equation ( 1 )$$

In Equation (1), A denotes a measurement point where blood pressure is measured when the arm is parallel to the shoulder (FIG. 7), P_{A} denotes an estimated hydrostatic pressure of blood at the measurement point A, h_{A} denotes a height from the ground, e.g., a level at which the user stands, to the measurement point A in the direction of gravity, e.g., vertically, B denotes a measurement point when the user's arm is raised up substantially straight, e.g., vertical or parallel to the direction of gravity, P_{B} denotes an estimated hydrostatic pressure of blood at the measurement point B, and h_{B} denotes a height from the ground to the measurement point B in the direction of gravity, e.g., substantially vertically from the ground to the measurement point B. At the measurement points A and B, a blood density is the same and is denoted as p, a flow rate of the blood is the same and is denoted as v, and acceleration due to gravity is the same and is denoted as g.

Equation (1) may manipulated, as shown in Equation (2), to calculate a difference between the estimated hydrostatic pressures at the measurement points A and B.

P_{A}+ρgh_{A}=P_{B}+ρgh_{B} Equation (2)

Thus, by manipulating Equation 2, the difference between the estimated hydrostatic pressures at the measurement points A and B, e.g., P_{A}−P_{B}, is determined by Equation (3).

P_{A}−P_{B}=ρg(h_{B}−h_{A}) Equation (3)

Accordingly, the difference between the estimated hydrostatic pressures at the two measurement points is calculated by multiplying the blood density, the acceleration due to gravity and the height difference between the two measurement points in the direction of gravity.

Thus, the hydrostatic pressure difference calculation unit 131 (FIG. 5) calculates the difference between the estimated hydrostatic pressures based on the height difference between the measurement points, the blood density and the acceleration due to gravity, stored in the storage unit 15.

Referring again to FIG. 5, the blood pressure difference calculation unit 132 acquires, from the blood pressure measurement unit 11, blood pressures measured at multiple measurement points having a height difference therebetween. The acquired blood pressures include, for example, a diastolic pressure, a systolic pressure and/or a mean blood pressure, as described in further detail above. It will be apparent to those of ordinary skill in the art that the blood pressure in an exemplary embodiment includes all information relating to pressure acting against wall of blood vessels as blood of a user flows through the blood vessels, wherein the pressure may be measured by using any measurement method described above (but not limited thereto).

The blood pressure difference calculation unit 132 calculates a difference between blood pressures measured at multiple, e.g., two, measurement points having a height difference therebetween, acquired by the blood pressure measurement unit 11. Once the blood pressures have been measured at the two measurement points, e.g., at measurement points A and B (FIG. 7), the blood pressure measured at measurement point A is subtracted from the blood pressure measured at measurement point B. In an exemplary embodiment, the blood pressures include the diastolic pressure and/or the systolic pressure measured at each measurement point, for example. The blood pressure difference is calculated using the diastolic pressure and/or the systolic pressure according to the user's selection. Specifically, when using the diastolic pressure, the diastolic pressure measured at the first measurement point A is subtracted from the diastolic pressure measured at the second measurement point B. In an exemplary embodiment, either the diastolic pressure or the systolic pressure is used. However, a reliability is increased when the diastolic pressure is used.

Still referring to FIG. 5, the error calculation unit 133 calculates an error by comparing a difference between the estimated hydrostatic pressures, calculated by the hydrostatic pressure difference calculation unit 131, and a difference between the measured blood pressures, calculated by the blood pressure difference calculation unit 132. Thus, the error is calculated by subtracting the lesser of a difference between the estimated blood pressure P_{E} and a difference between the measured blood pressures P_{M} from the larger of the difference between the estimated blood pressure P_{E} and the difference between the measured blood pressures P_{M}. More specifically, an operation performed in the error calculation unit 133 is shown mathematically in Equation (4).

(Error)=|P_{E}−P_{M}| Equation (4)

In Equation (4), P_{E} represents a difference between the estimated hydrostatic pressures, determined by the hydrostatic pressure difference calculation unit 131, P_{M} represents a difference between the measured blood pressures, determined by the blood pressure difference calculation unit 132, and “Error” indicates an error, e.g., an absolute value of a difference between P_{E} and P_{M}. In an exemplary embodiment, the error is displayed using the user interface unit 12 (best shown in FIG. 4).

Still referring to FIG. 5, the comparison unit 14 compares the error determined by the error calculation unit 133 with an allowable standard error and reports to the user, via the user interface unit 12, whether correction is required. Standards for approval of blood pressure measurement apparatuses by the U.S. Food and Drug Administration (“US FDA”) require that a difference between blood pressure measured using an auscultatory method with an upper arm cuff, and blood pressure measured using a corresponding blood pressure measurement apparatus shall be within a mean error of 5 mmHg, as suggested in Association for the Advancement of Medical Instrumentation (“AAMI”) SP-10: 2002. Therefore, if the error, e.g., the difference between P_{E} and P_{M}, is less than or equal to 5 mmHg, the comparison unit 14 determines that correcting the blood pressure monitoring apparatus 1 is unnecessary, and displays, via the user interface unit 12 that the blood pressure monitoring apparatus 1 is operating normally, e.g., within US FDA standards. In contrast, if the error calculated by the error calculation unit 133 is greater than 5 mmHg, the comparison unit 14 determines that correcting the blood pressure monitoring apparatus 1 is necessary, e.g., is required and displays, on the user interface unit 12, that correction of the blood pressure monitoring apparatus 1 is necessary. In an exemplary embodiment, methods of displaying whether correction is necessary include a visual method and/or an auditory method, as described in further detail above.

The storage unit 15 stores information used to determine whether correction of the blood pressure monitoring apparatus 1 is necessary. Information, such as the blood pressures measured by the blood pressure measurement unit 11, the blood density and physical size information of the user, for example, is also stored in the storage unit 15. In addition, an algorithm for controlling operation of the blood pressure monitoring apparatus 1, e.g., for the methods, functions, calculations and/or determinations described above is stored in the storage unit 15.

The correction unit 16 corrects the measured blood pressures based on the error calculated by the error calculation unit 133 and displays a result of the correction to the user via the user interface unit 12. Specifically, the correction unit 16 according to an exemplary embodiment corrects the measured blood pressures based on the error equivalent to the difference between the two calculated differences described above, e.g., one calculated by the hydrostatic pressure difference calculation unit 131 and the other calculated by the blood pressure difference calculation unit 132, and further corrects a correction formula used in the blood pressure monitoring apparatus 1. Thus, the correction unit 16 corrects the measured blood pressures, to substantially improve an accuracy thereof, by adding a value, equivalent to the error to the measured blood pressures, or, alternatively, by subtracting the value from the measured blood pressures. Alternatively, the correction unit 16 may correct the correction formula used by the blood pressure monitoring apparatus 1 itself.

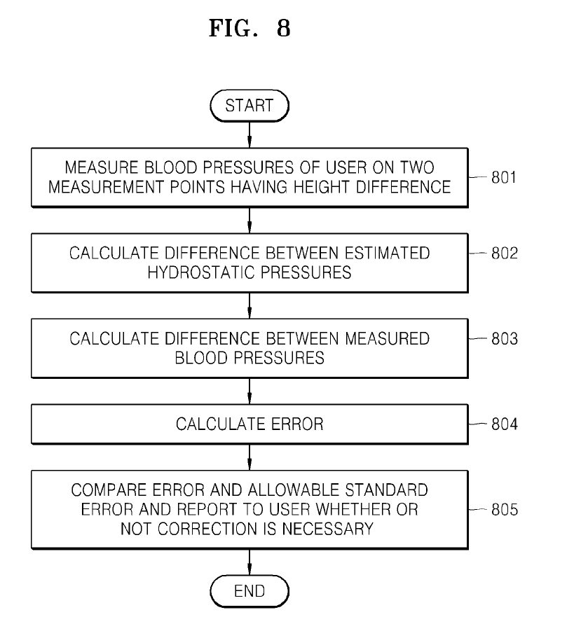

FIG. 8 is a flowchart illustrating an exemplary embodiment of a method of testing an accuracy of a blood pressure monitoring apparatus according to the present invention. Referring to FIG. 8, a method of testing the accuracy of a blood pressure measuring apparatus according to an exemplary embodiment includes operations performed sequentially in the blood pressure monitoring apparatus 1 (FIG. 5. It will be noted that, although not described separately herein, exemplary embodiments of a method of testing an accuracy a blood pressure monitoring apparatus applies to the exemplary embodiments of the blood pressure monitoring apparatus 1 described in further detail above.

In step 801, blood pressures of a user are measured at multiple, e.g., two, measurement points having a height difference therebetween. In an exemplary embodiment, measured blood pressures include a diastolic pressure and/or a systolic pressure measured at each of the two measurement points, but alternative exemplary embodiments are not limited thereto.

In step 802, the hydrostatic pressure difference calculation unit 131 (FIG. 5) calculates a difference between estimated hydrostatic pressures, based on the height difference between the two measurement points, a blood density and acceleration due to gravity. As described in further detail above, the height difference may be determined based on a height difference recognition sensor (not shown), an input from the user, or from an estimation based on a physical size of the user, for example. The blood density may determined from stored information, for example, and may vary according to the user's selection.

In step 803, the blood pressure difference calculation unit 132 (FIG. 5) calculates a difference between the measured blood pressures from the user measured in step 801. Either the diastolic pressure or, alternatively, the systolic pressure may be acquired according to the user's selection or depending on a configuration of the blood pressure monitoring apparatus 1. More specifically, an absolute value of a result of subtracting a blood pressure measured at a first measurement point A (FIG. 7) from a blood pressure measured at a second measurement point B (FIG. 7) is acquired. In an alternative exemplary embodiment, the blood pressures may be a diastolic pressure, a systolic pressure and/or an average blood pressure. In an exemplary embodiment, the types of blood pressures used to calculate the difference between the blood pressures measured at the first and second measurement points are the same. For example, when the diastolic pressure is used for the first measurement point, the diastolic pressure is also used for the second measurement point.

In step 804, the error calculation unit 133 calculates an error. As described above, the error calculated by the error calculation unit 133 is an absolute value of the result of subtracting a difference between the measured blood pressures, calculated in step 803, from a difference between the estimated hydrostatic pressures calculated in step 802.

In step 805, the error is compared with an allowable standard error, and whether correction of the blood pressure monitoring apparatus 1 is necessary, e.g., is required, is reported to the user. More particularly, when the error calculated in step 804 is less than or equal to 5 mmHg, which is an example of an allowable standard error for approval of blood pressure measurement apparatuses by the US FDA, it is determined that correction is unnecessary, e.g., is not required. However, if the error exceeds 5 mmHg, it is determined that correction is necessary, e.g., is required. A result of the determination of whether correction is reported to the user via the user interface unit 12 (FIG. 5).

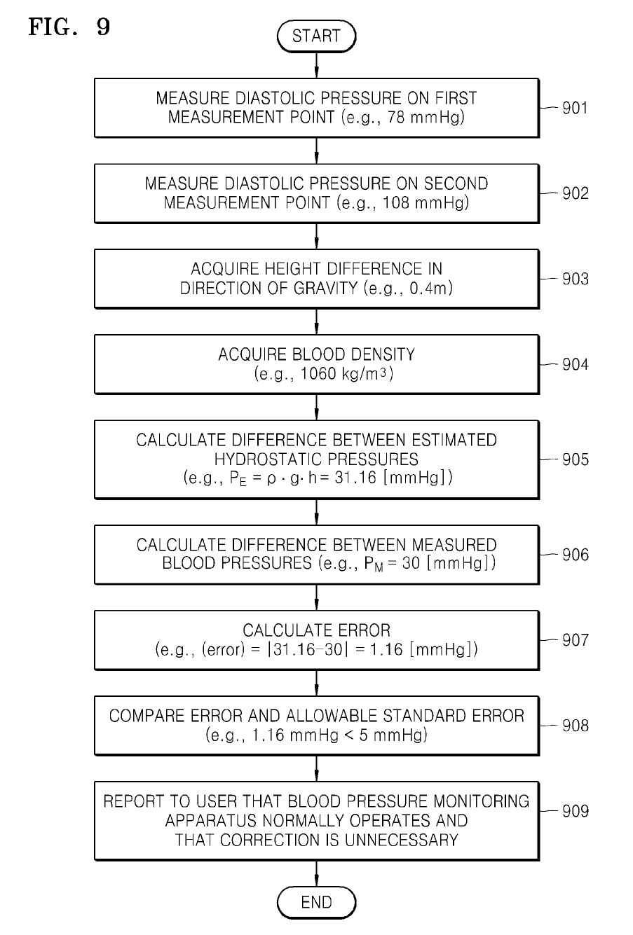

FIG. 9 is a flowchart illustrating an alternative exemplary embodiment of a method of testing an accuracy of a blood pressure monitoring apparatus according to the present invention.

In step 901, the blood pressure measurement unit 11 of the blood pressure monitoring apparatus 1 measures blood pressure at a first measurement point. In an exemplary embodiment, the blood pressure measurement unit 11 measures a diastolic blood pressure at the first measurement point and stores the diastolic pressure measured at the first measurement point in the storage unit 15 (FIG. 5) as a first blood pressure. The first measurement point may be measurement point A shown in FIG. 7, e.g., a measurement point where blood pressure is measured while the user horizontally stretches their arm to a height of their shoulder, as described in further detail above. Thus, when the diastolic pressure measured at the first measurement point is 78 mmHg, for example, the first blood pressure is stored in the storage unit 15 is 78 mmHg.

In step 902, the blood pressure measurement unit 11 of the blood pressure monitoring apparatus 1 according to an exemplary embodiment measures blood pressure at a second measurement point. Specifically, the blood pressure measurement unit 11 measures a diastolic blood pressure at the second measurement point, and stores the diastolic pressure measured at the second measurement point in the storage unit 15 as a second blood pressure. The second measurement point may be measurement point B (FIG. 7), for example, wherein blood pressure is measured while the user raises their arm straight up. When the diastolic pressure measured at the second measurement point is 108 mmHg, for example, the secondary blood pressure stored in the storage unit 15 is 108 mmHg.

In step 903, the hydrostatic pressure difference calculation unit 131 of the blood pressure monitoring apparatus 1 acquires a height difference between the two measurement points. As described in greater detail above, the height difference may be determined, for example, from a height difference recognition sensor (not shown), from a user input, or as a result of an estimation based on a physical size of the user. For example, when the height difference is acquired from the height difference recognition sensor and the height difference between the first and second measurement points is 40 cm, the hydrostatic pressure difference calculation unit 131 acquires a value of 0.4 m as the height difference.

In step 904, the hydrostatic pressure difference calculation unit 131 of the blood pressure monitoring apparatus 1 acquires a blood density from the storage unit 15 or, alternatively, from the user input. For example, when a blood density value stored in the storage unit 15 is used, the hydrostatic pressure difference calculation unit 131 may acquire a blood density value of 1060 kg/m³ as the blood density, but alternative exemplary embodiments are not limited thereto.

In step 905, the hydrostatic pressure difference calculation unit 131 calculates a difference between the estimated hydrostatic pressures based on the height difference, the blood density and the acceleration due to gravity. As described in greater detail above, the difference between the estimated hydrostatic pressures may be determined by multiplying the height difference, the blood density and the acceleration due to gravity. In an exemplary embodiment, the difference between the estimated hydrostatic pressures may be calculated using Equation (5), for example.

P_{E}=1060[kg/m³]×9.8[m/s²]×0.4[m]=4155.2[Pa]=31.16[mmHg] Equation (5)

In step 906, the blood pressure difference calculation unit 132 calculates a difference between the measured blood pressures based on information about the blood pressures measured by the blood pressure measurement unit 11 and stored in the storage unit 15. For example, a difference between the first blood pressure and the second blood pressure measured above is 30 mmHg. Therefore, the difference between the measured blood pressure differences is 30 mmHg.

In step 907, the error calculation unit 133 calculates a difference between the calculated differences from the hydrostatic pressure difference calculation unit 131 and the blood pressure difference calculation unit 132. For example, in an exemplary embodiment, when the difference between the estimated hydrostatic pressures is 31.16 mmHg and the difference between the measured blood pressures is 30 mmHg, the error is 1.16 mmHg, as shown in FIG. 9.

In step 908, the error calculated in step 907 is compared with an allowable standard error. In an exemplary embodiment, the allowable standard error may be in a range less than or equal to 5 mmHg, but the allowable standard error may be varied according to the user's selection, for example. As shown in FIG. 9, the error is 1.16 mmHg, which is within the allowable standard error range.

In step 909, whether correction of the blood pressure monitoring apparatus 1 is necessary is determined and is reported to the user, as is whether the blood pressure monitoring apparatus 1 is operating normally, e.g., within the allowable standard error range. Thus, if the error is within the allowable standard error range, it is determined that correction is unnecessary. Conversely, if the error is not within the allowable standard error range, it is determined that correction is necessary. The result of the determination is reported to the user, via the user interface unit 12 (FIG. 5). In the exemplary embodiment shown in FIG. 9, for example, the error is within the allowable standard error range, and it is thereby determined that correction is unnecessary, and the result of the determination is displayed on the user interface unit 12.

As described herein, according to exemplary embodiments, it is determined, without an additional apparatus, whether there is a need to correct a blood pressure measurement apparatus. Thus, in a case of a noninvasive, nonintrusive, continuous blood pressure measurement method convenient to use but less accurate, a reliability of measured blood pressures is substantially increased by determining whether there is a need to correct the blood pressure measurement apparatus by simply performing the blood pressure measurement twice. In addition, a need to dispatch blood pressure measurement apparatuses to respective manufacturers periodically, e.g., at least once every two or three years, for error correction, the blood pressure monitoring apparatus 1 according to an exemplary embodiment accurately determines whether correction is necessary (and then corrects the error if necessary) thereby substantially reducing time and cost required for the correction.

In addition, alternative exemplary embodiments include computer readable code/instructions in/on a medium, e.g., a computer readable medium, to control at least one processing element to implement any or all of above described exemplary embodiments. In addition, the medium includes any medium/media permitting storage and/or transmission of the computer readable code/instructions.

Moreover, the computer readable code can be recorded/transferred to the medium in a variety of ways, such as exemplary embodiments wherein the medium includes recording media, such as magnetic storage media (e.g., read only memory (“ROM”), floppy disks, hard disks, etc.), as well as optical recording media (e.g., compact disc-ROMs (“CD-ROMs”), and/or digital versatile discs (“DVDs”), and transmission media such as media carrying or including carrier waves, as well as elements of the Internet. Thus, the medium according to an exemplary embodiment may be a defined and measurable structure including and/or carrying a signal or information, such as a device carrying a bitstream, for example. The media may also be a distributed network, so that the computer readable code is stored/transferred and/or executed in a distributed fashion. Furthermore, the processing element according to an exemplary embodiment includes, for example, a processor or a computer processor, and processing elements may be distributed and/or included in a single device.

The present invention should not be construed as being limited to the exemplary embodiments set forth herein. Rather, these exemplary embodiments are provided so that this disclosure will be thorough and complete and will fully convey the concept of the present invention to those skilled in the art.

While the present invention has been particularly shown and described with reference to exemplary embodiments thereof, it will be understood that the exemplary embodiments described therein should be considered in a descriptive sense only and not for purposes of limitation. Moreover, it will be understood by those of ordinary skill in the art that various changes in form and details may be made to the exemplary embodiments described herein without departing from the spirit or scope of the present invention as defined by the following claims.

What is claimed is:

1. A method of testing accuracy of blood pressure measurement in a blood pressure monitoring apparatus, the method comprising:
calculating a difference between measured blood pressures of a user measured at two or more measurement points;
calculating a difference between hydrostatic pressures of blood estimated at the two or more measurement points based on a height difference between the two or more measurement points and a blood density; and
calculating an error of the measured blood pressures based on the difference between the measured blood pressures and the difference between the hydrostatic pressures of blood.

2. The method of claim 1, further comprising:
comparing the error to an allowable standard error; and
reporting to the user at least one of whether there is a need to correct the blood pressure monitoring apparatus and whether the blood pressure monitoring apparatus is operating normally.

3. The method of claim 1, wherein the calculating of the difference between the measured blood pressures comprises calculating a difference between a first blood pressure measured at a first measurement point and a second blood pressure measured at a second measuring point at a different height than the first measurement point.

4. The method of claim 2, wherein the allowable standard error is inputted by the user.

5. The method of claim 1, wherein the height difference between the two or more measurement points is estimated based on a physical size of the user.

6. The method of claim 1, wherein
the calculating of the difference between the hydrostatic pressures of blood comprises using at least one of the height difference between the two or more measurement points and the blood density, and
the at least one of the height difference between the two or more measurement points and the blood density is inputted by the user.

7. The method of claim 1, further comprising correcting the measured blood pressures based on the error.

8. A computer program product comprising:
a computer readable computer program code for implementing a method of testing accuracy of blood pressure measurement in a blood pressure monitoring apparatus; and
instructions for causing a computer to implement the method, the method comprising:
calculating a difference between measured blood pressures of a user measured at two or more measurement points;
calculating a difference between hydrostatic pressures of blood estimated at the two or more measurement points based on a height difference between the two or more measurement points and a blood density; and
calculating an error of the measured blood pressures based on the difference between the measured blood pressures and the difference between the hydrostatic pressures of blood.

9. A blood pressure monitoring apparatus comprising:
a blood pressure measurement unit which measures blood pressures of a user at more than two measurement points to generate measured blood pressures;
a blood pressure difference calculation unit which calculates a difference between the measured blood pressures;
a hydrostatic pressure difference calculation unit which calculates a difference between hydrostatic pressures of blood estimated at the more than two measurement points;
an error calculation unit which calculates an error based on the difference between the measured blood pressures and the difference between the hydrostatic pressures of blood; and
a user interface unit which reports the error to the user.

10. The blood pressure monitoring apparatus of claim 11, further comprising a comparison unit which compares the error and an allowable standard error, wherein the user interface unit reports to the user at least one of whether there is a need to correct the blood pressure monitoring apparatus and whether the blood pressure monitoring apparatus is operating normally, based on a result of the comparison of the error and the allowable standard error by the comparison unit.

11. The blood pressure monitoring apparatus of claim 12, wherein
the user interface unit is configured to acquire at least one of a height difference and blood density from the user, and
the hydrostatic pressure difference calculation unit calculates the difference between the hydrostatic pressures of blood based on the at least one of the height difference and the blood density acquired by the user interface.

12. The blood pressure monitoring apparatus of claim 13, wherein
the user interface unit is further configured to acquire the allowable standard error from the user.

13. The blood pressure monitoring apparatus of claim 11, further comprising a correction unit which corrects the measured blood pressures based on the error.
